(19) United States
(12) Patent Application Publication
(10) Pub. No.: US 2008/0020477 A1
(43) Pub. Date: Jan. 24, 2008

(54) Salivary Glucose Monitoring

(76) Inventor: Allan D. Pronovost, San Diego, CA (US)

Correspondence Address: FOLEY & LARDNER LLP, 111 HUNTINGTON AVENUE, 26TH FLOOR, BOSTON, MA, 02199-7610, US

(21) Appl. No.: 11/574,952

(22) PCT Filed: Sep. 12, 2005
(86) PCT No.: PCT/US05/32466

§ 371 Date: Aug. 29, 2007

Related U.S. Application Data
(60) Provisional application No. 60/608,796, filed on Sep. 10, 2004.
(60) Provisional application No. 60/608,679, filed on Sep. 10, 2004.
(60) Provisional application No. 60/609,388, filed on Sep. 13, 2004.

(51) Int. Cl. G01N 33/00; C12M 1/00

(52) U.S. Cl. 436/95; 73/61.43

(57) ABSTRACT

The present invention relates to the measurement of carbohydrate in a fluid and uses thereof. Specifically, the invention is directed to the field of glucose measurement in the saliva of a subject. The invention provides devices and mathematical algorithms for the measurement of glucose in a subject.

### FIELD OF THE INVENTION

The present invention relates to the measurement of carbohydrate in a fluid and uses thereof. Specifically, the invention is directed to the field of glucose measurement in the saliva of a subject. The invention discloses devices and mathematical algorithms for the measurement of glucose in a subject.

### BACKGROUND

Saliva contains a variety of components that will actively interfere with salivary glucose monitoring over time following collection of either non-stimulated or stimulated saliva after appropriate fasting. U.S. Pat. No. 6,102,872, U.S. Pat. No. 4,817,632 and WO 00/64 334 describe the use of osmotic driver and time (20 min) for the in situ equilibrium dialysis of glucose in saliva for subsequent processing and detection. The methodology employs a double membraned, sealed, dialysis sac (saliva sac) that is placed in the mouth to equilibrium dialyze saliva over time on a passive basis. Various means are described so as to access the processed saliva in the sac with visually read, enzymatic calorimetric (non-electrochemical) screening or monitoring means that do not utilize instrumentation (such as a monitor i.e., potentiostat) to quantitatively measure glucose.

The limitations to the technology in this patent are numerous. The sac has to be a sealed sac to allow osmotic driver contained in the sac to work to force fluid into the sealed sac as this does not naturally enter. This equilibrium dialysis takes 20 minutes to complete at a minima if excess osmotic driver is utilized; times less than that result in too much driver remaining in the sac which interferes with the measurement of glucose. Osmotic driver delivered to the mouth over time has an unpleasant taste, may be toxic, interferes with glucose levels as stimulation reoccurs and excess salivary fluid dilutes initial stimulated or non-stimulated glucose values. The saliva sac is difficult at best to seal making manufacturing a problem. The sealants described and used for sealing sacs are toxic and the chemicals may cause cancer in some individuals. Sacs loose elasticity and filter quality over long-term storage. Once collected, the sac has to be carefully opened as contents are usually under pressure, which prohibits design of a reliable all in one device as proposed. Another issue observed is the glycerol used to keep the membrane supple over time to promote shelf life actively interferes with glucose measurement and glucose values determined need to be corrected for this interferent which can vary sac to sac and which prohibits real time monitoring. The sac is inconvenient from a consumer standpoint in that it induces a gag reflex. Some patients are also allergic to sac components or additives. The sac is a laboratory method not ready for use as a medical device as described.

WO 003007814 describes a transport system for holding glucose in a suspended state within the sample that utilizes the sequestration (hiding) of glucose within the sample through a process of molecular adsorption within a gel matrix with a MW fractionation range of <1,500 daltons. This facilitates the transport of the non-separated sample (over 5 days) to a centralized laboratory for subsequent processing and glucose detection using expensive laboratory instrumentation. At the laboratory, the adsorbed glucose is only released from the gel matrix by reverse ion exchange under harsh reverse elution conditions requiring sample dilution after elution to allow detection by only an expensive electrochemical glucose sensor instrument. The patent application also refers to the use of differential adsorption using an adsorption matrix with a molecular weight fractionation range above glucose to allow glucose to travel through unimpeded in the void volume while MW materials above the lower limit of the adsorptive range are retained. The materials described for such use are gel filtration media. But subsequent review of the gel filtration media chromatography literature from the supplier of the gel supplier cited in the application clearly indicate that all material that enters into the gel matrix do indeed get trapped within the matrix and are separated by size chromatography methods wherein the smallest MW material indeed elutes well after the high MW material, and not in the void volume as stated in the application. Only interstitial fluid comprises the void volume. Hence the “pass through” feature described in the patent is in scientific error.

All of the patents cited above rely on the passive separation of glucose from salivary material based on passive physical methods or means such as dialysis or osmosis. No attempt is made to remove, process, or deal with materials present in saliva that actually interfere with saliva detection. As such, the patents describe procedures that are passive in nature not relying on principles that directly address the real issue of glucose detection in mixed whole saliva when glucose availability for detection is masked by salivary components. As such the procedures described are non-specific, slow, generally ineffective and try to bypass the issue in its entirety. This is evidenced by the relatively poor correlations observed for saliva relative to whole blood noted in the applications wherein responses obtained by such methods are not quantitative for monitoring but quantal (only 2 cutoffs for 2 hour fasting were obtained, negative and diabetic; and only 3 cutoffs for 8 hour fasting were obtained, negative—impaired—diabetic). These quantal cutoffs offer insufficient precision for monitoring purposes and are only suitable for screening applications.

There remains a need for improved means of measuring salivary glucose.

### SUMMARY OF THE INVENTION

The invention provides for various devices and methods of processing a saliva sample obtained from a mammal, particularly a human or a companion animal such as a dog, horse or cat. The saliva sample is processed and the carbohydrate content of the saliva can be determined. Salivary carbohydrate levels reflect and relate to blood carbohydrate levels, and can be used to predict a predisposition for, or to indicate treatment of a disorder characterized by elevated or low blood glucose levels, such as diabetes.

In one aspect, the invention provides a method of determining salivary glucose levels in a mammal comprising: obtaining a sample of saliva from the mammal, processing the sample thereby substantially purifying the saliva, and analyzing the processed sample for the presence of soluble carbohydrates, wherein a quantity of salivary carbohydrates in the processed sample correlates with blood carbohydrate levels in the mammal. In one embodiment, processing the sample further comprises filtering the sample to partition low molecular weight analytes from high molecular weight contaminants and particulate matter. In another embodiment, filtration is accomplished through axially directed migration of the sample through tightly packed axially aligned fibers. In still another embodiment, filtration is accomplished through one or more nanopore membranes, the nanopore membranes having a median pore diameter from about 200 nanometers to about 2 nanometers. In yet another embodiment, the method further comprises removing proteins from the processed sample. In still another embodiment, proteins are adsorbed to a substrate. In even still another embodiment, the substrate is nitrocellulose, nylon or polyvinylidene fluoride. In one embodiment, the method further comprises absorbing glucose from the processed sample. In another embodiment, glucose is absorbed to a substrate consisting of porous absorbents having an internal surface area greater than about 400 M2/gram. In still another embodiment, glucose is absorbed to a substrate selected from the group consisting of: a zeolite, aluminum oxide microspheres, ceramic microspheres, hydrous alumina silicate microspheres, alumina dessicant beads, attapulgus clay beaded silica gel dessicants, natural clay absorbents, and activated carbon.

The method described is useful particularly where the mammal is afflicted with a disorder characterized by aberrant levels of blood carbohydrates, such as diabetes. In specific embodiments, the quantities of salivary carbohydrates obtained from the processed sample indicate an appropriate therapeutic insulin dosage for treating the disorder. In other embodiments, the mammal is preconditioned prior to obtaining the sample of saliva by being provided with a compound capable of stimulating the production and let down of saliva in the mammal.

In another aspect, the invention provides a device for processing saliva comprising: a saliva sample introduction port, a filter, and an absorbent matrix, wherein a sample of saliva is processed to remove high molecular weight contaminants and glucose in the processed saliva is absorbed to the matrix. In one embodiment, the filter comprises tightly packed axially aligned fibers. In one embodiment, the filter comprises one or more nanopore membranes, the nanopore membranes having a median pore diameter from about 200 nanometers to about 2 nanometers. In another embodiment, the device further comprises a substrate capable of irreversibly binding proteins in the saliva sample, such as nitrocellulose, nylon or polyvinylidene fluoride. In another embodiment, the device includes a glucose absorbent substrate. In one embodiment, the glucose absorbent substrate consists of porous absorbents having an internal surface area greater than about 400 M2/gram. In yet another embodiment, the device includes a glucose absorbent substrate selected from the group consisting of: a zeolite, aluminum oxide microspheres, ceramic microspheres, hydrous alumina silicate microspheres, alumina dessicant beads, attapulgus clay beaded silica gel dessicants, natural clay absorbents, and activated carbon. In another embodiment, the device further comprises a sensor for detecting glucose levels in the processed saliva sample. In another embodiment, the device further comprises a processor, wherein the processor correlates salivary carbohydrate levels in the sample with reference blood carbohydrate levels thereby calculating a range of probable blood carbohydrate levels based on the saliva sample carbohydrate levels, and having an output for displaying information calculated by the processor. In another embodiment, the device further comprises a processor which correlates salivary carbohydrate levels of a user of the device with historical blood carbohydrate levels or historical salivary carbohydrate levels of the user of the device. In another embodiment, the processor correlates salivary carbohydrate levels of a user of the device with historical medical or lifestyle information of the user of the device. In another embodiment, the processor correlates salivary carbohydrate levels of a user of the device with genetic information about the user of the device. In still another embodiment, the device includes an output that displays information indicating an appropriate therapeutic insulin dosage for the user based on the salivary glucose levels detected in the mammal.

### BRIEF DESCRIPTION OF THE DRAWINGS

FIG. 1 is a schematic drawing illustrating an embodiment of the device of the invention. The device includes a squeeze bulb 101 that can be articulated through depression of the top 107 and bottom 108 sides. Saliva is introduced through a port 109 and is drawn through a first filter 104, a second filter 105 and a third protein absorption membrane 106 to remove cellular debris, large molecular weight molecules and proteins as described. The resultant processed saliva contains low weight molecules and glucose. Removal of the cap 103 allows the processed salivary fluid 102, to be withdrawn through a port 110.

FIG. 2 is a schematic drawing illustrating a second embodiment of the device of the invention. The device includes a squeeze bulb 201 that can be articulated through depression of the left 224 and right 221 sides. Saliva is introduced through a port 223 and is drawn through a first filter 207, a second filter 206 and a protein absorption membrane 205 to remove cellular debris, large molecular weight molecules and proteins as described. A floating ball in one way valve 204 is shown. The resultant processed saliva contains low weight molecules and glucose. Removal of the cap 203 allows the processed salivary fluid 202, to be withdrawn through a port 222.

FIG. 3 is a schematic drawing illustrating a third embodiment of the device of the invention. A squeeze barrel 305 design is shown. A saliva sample is introduced into a port 334, and is drawn into the device through vacuum resulting from articulation of the top 331 and bottom 333 of the squeeze barrel 305. The saliva sample is processed through sequential filtration 301 and 303 devices and a protein absorption membrane 304. The processed saliva 302 is retained in the tip junction 307, until the twist off disposable tip 306 is removed, at which time the saliva can be dispensed upon inversion of the device and by articulation of the squeeze barrel 305.

FIG. 4 is a schematic drawing illustrating a fourth embodiment of the device of the invention. FIG. 4a shows a cutaway schematic of the device, and FIG. 4b shows a side view of the device in a closed configuration. In FIG. 4a, the device as illustrated has an articulatable lid 415. A saliva sample is introduced into the lumen 441 of the device. Processing of the sample occurs through sequential filtration through a first filter 401 and a second filter 402. Protein absorption to a third membrane 403 renders the saliva sample substantially free of high molecular weight substances and proteins. In FIG. 4b, the sample is introduced into the device and the top 415 is closed via a hinge mechanism 417. Articulation of the top of the device 416 forces the sample through the filtration mechanisms and the processed saliva sample 420 flows out through a channel 450 in the bottom of the device.

FIG. 5 is a schematic drawing illustrating a fifth embodiment of the device of the invention. The device provides an aperture 551 defining the opening of a well 508 into which a user expectorates a saliva sample 502. The well 508 is integral with a top housing 509 and a bottom housing 510 of the device. Proximal to the well 508, filtration devices 504 and 503 remove the cellular debris and large molecular weight proteins. A protein binding membrane 501 traps proteins and provides a wick that draws the processed saliva sample through the housing 509 and 510. An opening in the housing 512 provides a point of insertion 552 for a sensor strip 511. In various embodiments, the sensor strip may provide for entrapment of the processed saliva sample or for absorption of glucose from the processed saliva sample.

FIG. 6 is a schematic drawing illustrating a sixth embodiment of the device of the invention. FIG. 6a shows an inverted side view of the device. FIG. 6b shows a noninverted side view of the device. The device has top 609 and bottom 610 housing members. A port 603 allows introduction of the saliva sample. Filtration is accomplished by a first filtration device 602. Protein absorption follows, as the filtered sample contacts a protein immobilization membrane 601, and further provides a wicking action that draws the processed saliva sample through the housing. An lumen in the housing 612 is adapted to receive a sensor strip 611, through an opening 661. In various embodiments, the sensor strip may provide for entrapment of the processed saliva sample or for absorption of glucose from the processed saliva sample.

FIG. 7 is a schematic drawing illustrating a seventh embodiment of the device of the invention. The device has top 709 and bottom 710 housing members. A port 703 allows introduction of the saliva sample. Filtration is accomplished by a first filtration device 702. Protein absorption follows, as the filtered sample contacts a protein immobilization membrane 701 and further provides a wicking action that draws the processed saliva sample through the housing. An lumen in the housing 712 is offset from the terminal end of the protein binding membrane 701, and the lumen 712 is adapted to receive a sensor strip 711, through an opening 771. In various embodiments, the sensor strip may provide for entrapment of the processed saliva sample or for absorption of glucose from the processed saliva sample.

FIG. 8 is a schematic drawing illustrating an eighth embodiment of the device of the invention. FIG. 8a shows the device as a whole having a body 814 and a filtration assembly 803. FIG. 8b shows the terminal end of the device wherein the filtration assembly 803 is shown in greater detail. FIG. 8c shows the device in cross section. The device has top 809 and bottom 810 housing members. The saliva sample is applied to the terminus 805 of a first filtration device 803, which wicks the sample and removes high molecular weight contaminants. Further filtration is accomplished by a second filter 801. Protein absorption follows, as the filtered sample contacts a protein immobilization membrane 802, which further provides a wicking action that draws the processed saliva sample through the housing. An lumen in the housing 812 is offset from the terminal end of the protein binding membrane 802, and the lumen 812 is adapted to receive a sensor strip 811, through an opening 881. In various embodiments, the sensor strip may provide for entrapment of the processed saliva sample or for absorption of glucose from the processed saliva sample.

FIG. 9 is a graph illustrating the relationship of nanoamps to mg/dL values in saliva for the patients studied.

FIG. 10 is a graph illustrating the relationship of saliva glucose level to blood glucose level in clinical samples.

It will be realized by a skilled artisan that the various devices disclosed can provide for a combination of filtration and absorption means, and can employ various active or passive flow methodologies. Accordingly the above embodiments are considered nonlimiting examples only.

### DETAILED DESCRIPTION OF THE INVENTION

General

The present invention relates to the measurement of carbohydrate in a fluid and uses thereof. Specifically, the invention is directed to the field of glucose measurement in the saliva of a subject. The invention discloses devices and mathematical algorithms for the measurement of glucose in a subject.

Saliva contains a variety of components that will actively interfere with salivary glucose monitoring over time following collection of either non-stimulated or stimulated saliva after appropriate fasting.

Saliva is a viscous, dense, sticky fluid innately containing microorganisms like bacteria and fungi, intact human cells, cellular debris, and many soluble materials. Some of the factors that can effect glucose detection and monitoring in saliva include: the enzymatic degradation of glucose (by enzymes normally found in the mouth); degradation of glucose by microbes wherein glucose is a food source; host cellular metabolism for energy; adherence of glucose to mucins, polysaccharides, and proteinaceous materials; and the inherent molecular instability of the glucose molecule itself over time owing to isomerization and other intramolecular variations (glucose exists in a left and right form, the ratio of which can vary spontaneously; glucose also converts depending upon pH and ionic strength to other isomeric forms such as fucose and mannose; glucose also changes structural form based on rotation around anomeric carbon 2). The present invention solves this problem by affording the means to actively circumvent these detrimental factors to facilitate glucose processing for monitoring.

With the aim of true monitoring using saliva, and owing to the limitations cited in the prior art, an improved salivary glucose processing means for monitoring can afford some, or all, of the following features in some embodiments:
Immediate removal of stimulated mixed whole saliva from the mouth cavity to avoid ductal resorption or cellular metabolism
No interference by solubilized salt osmotic drivers or dialysis membrane surfactant softening agents (glycerol) to facilitate such removal without alteration
No time-dependent collection requirement to reach equilibrium (20 min) after stimulation
Immediate and efficient active processing and delivery of glucose from stimulated whole saliva to the detection means
Immediate (instantaneous) detection of glucose within the sample liquid upon delivery to the sensor means without the need for further sample elution or processing
Detection by an electrochemical sensor system with sufficient sensitivity and resolution to measure the lower levels of glucose found in salivary fluid

In one aspect, the present invention provides various “combinations of integrated active processes” that collectively (in varying combinations dependent upon collection device designs) allow for the efficient collection, processing and delivery of glucose from stimulated or non-stimulated mixed whole saliva for detection by a sufficiently sensitive electrochemical sensor strip and associated instrument detection means so as to allow salivary glucose detection to be used as a substitute for finger stick blood detection of glucose.

Saliva Multifunctionality and Heterogeneity

Saliva is a heterogeneous fluid whose composition changes based on its multifunctionality. It is a dynamic media that can change drastically based on the functional need of the individual. Monitoring of glucose in saliva necessitates an understanding of the dynamic nature of saliva and the development of an active processing method for saliva glucose monitoring requires control of the extremes that may be encountered in diabetics undergoing monitoring on a routine basis. As such the molecular heterogeneity of saliva is described below.

Salivary fluid exhibits various functions. Attributable to each function are soluble molecular components that are secreted by the body to actively afford saliva those specific properties. Effective saliva processing for glucose monitoring necessitates dealing with these soluble factors to remove them as interfering substances that serve to make salivary glucose detection and monitoring difficult at best.

Saliva exhibits the following functions (materials secreted shown in parentheses): lubrication and viscoelasticity (mucins, statherins); tissue coating (amylases, cystatins, mucins, proline rich proteins, statherins); mineralization (cystatins, histatins, proline rich proteins, statherins); digestion (amylases, mucins, lipase); buffering (carbonic anhydrases, histatins); and antimicrobial activity (mucins, peroxidases, lysozyme). As such the major secreted soluble salivary components can be rank ordered based on approximate MW as follows: mucin 1 (1,000 kDa), slgA (600 kDa), mucin 2 (150 kDa), IgG (140 kDa), lactoferrin (90 kDa), peroxidases (85 kDa), amylases 80 kDa), carbonic anhydrase (70 kDa), proline rich proteins (50 kDa), lysozyme (20 kDa), statherins (7 kDa), and histatins (3 kDa).

Mucus is produced by the biosynthetic activity of secretory cells. Mucus molecules are able to join together to make polymers or occur as an extended 3 dimensional network (gel). Mucus is glycoprotein in nature. slgA and IgG are ‘protein’-based immunoglobulins. Amylases are protein-based enzymes that hydrolyze alpha 1-4 bonds of starches such as amylose and amylopectin. Lingual lipase is a protein enzyme secreted by the von Ebner's glands of the tongue and is involved in fat digestion. Statherins as proteins prevent precipitation of supersaturated calcium phosphate in saliva to maintain tooth enamel. Proline rich proteins (PRP's) present in saliva inhibit calcium phosphate crystal growth. Lysozyme is a protein enzyme secreted by the salivary glands which has antimicrobial activity. Histatins are histidine rich proteins that are potent inhibitors of Candida albicans growth. C. albicans is a common oral yeast infection in diabetics. Cystatins are protein based inhibitors of cysteine proteases found in oral fluid. Sialoperoxidase (salivary peroxidase) is a protein-based enzyme with antimicrobial activity. Myeloperoxidase, a protein enzyme from leukocytes is commonly found in saliva as well.

It is important to note that the above soluble interfering materials are all ‘protein’ in nature; either as protein, glycoprotein, lipoprotein, or the like. One of the processes used below affords the use of that protein constituency as the basis for active removal of all of these protein-based substances from saliva. Aside from the above described soluble proteinaceous materials, saliva may also carry a varying types of insoluble materials. These can include overt particulate material, colloidal gel-like material, globular or polymeric macromolecular material (these items may be fully insoluble, semi-soluble, or exist as colloid). Examples include intact or lysed bacteria or fungal cells, intact host cells, leukocytes or erythrocytes, lysed host cells, intracellular materials and organelles, nucleic acid from host or prokaryotic sources, and the like. Different processes as described below will actively remove these particulate and insoluble materials.

As such saliva is a dynamic heterogeneous fluid that varies in composition over time. It contains a variety of materials that may be found in particulate (particle) form, macromolecular form, gel form, soluble or insoluble polymers (mucin or DNA), or soluble protein containing materials. Each of these materials can be actively eliminated, reduced or minimized using different processes for the purpose of salivary glucose monitoring by electrochemical instrumented means. This is accomplished through a combination of active processes integrated into a disposable saliva collection and processing device. Description of such active processes and their integration into various types of saliva glucose collection devices is the basis of this invention.

It is important for sake of the use of saliva for monitoring, to note up front that saliva is very useful if it is used as a non-invasive fluid following abstinence from sugar containing food and drink for at least 2 hours. It is well-established that fasting an appropriate time period (2-8 hours) before saliva monitoring minimizes the occurrence of trace foodstuffs. This limits the use of saliva alone or as an adjunct to blood for testing >2 hrs after food consumption. Suitable times for diabetic monitoring include upon rising; immediately before lunch or dinner; or mid-morning, mid-afternoon, or >mid-evening after abstinence from food or sugar containing drink for >2 hrs. Before meals are often the time diabetics test themselves to assess their baseline values and not immediately after eating.

Active Integrated Processes of the Invention

The present invention provides various combinations of active integrated processes that collectively allow for the efficient collection, processing and delivery of glucose from stimulated or non-stimulated mixed whole saliva for detection by electrochemical sensor strip and instrument detection means. The individual processes and the combinations of processes described herein work both individually and in concert to facilitate the active removal of various types of interfering substances from saliva, namely two main types—1. insoluble particulate, or 2. soluble material; both of which are naturally present in saliva. In addition, the individual processes and the combinations of processes described herein are designed to facilitate delivery of a sufficient volume of processed salivary fluid (containing glucose) to the electrochemical sensor strip detection means for subsequent quantitation. Combinations of processes are integrated into saliva collection devices whose construction and design facilitate the seamless integration of processes into a one-step device.

Suitable samples for salivary glucose monitoring using the one-step devices described herein comprise unstimulated or stimulated mixed whole saliva. Saliva samples are collected using one of the collection means described herein immediately after stimulation and tested with the sensor strip within 15 minutes of processing for best results.

Suitable methods for stimulation exist in the art. These include physical (mastication), chemical (citrate, tartrate), olfactory, or mental stimulation means. For example, certain sigma ligands can be effective systemic secretagogues, and therefore, used to effectively treat dry mouth, see U.S. Pat. No. 5,387,614. Likewise, U.S. Pat. No. 4,088,788 discloses stimulation of saliva production by the use of at least three percent by weight of an organic acid selected from the group consisting of adipic, ascorbic, citric, fumaric, lactic, malic and tartaric acids, and saccharin. The organic acid and saccharin combination provides a synergistic saliva stimulating effect. Further synergistic effects are provided by combining a high level of dextrose with the organic acid to improve the hygroscopicity and shelf life, but the added sugar is contraindicated for use by diabetic patients. The preferred stimulant is approximately 20 mg of citric acid, administered orally, such as sublingually. Delivery of the stimulant can be in powder form (in a sealed cellopack), or can be coated on the portion of the collection device placed in the mouth, or can be supplied as a small, tart candy, preferably sugar-free and suitable for administration to a diabetic patient. If coated onto the collection device, the citric acid can be mixed with a variety of soluble dispersants known in the art and allowed to dry after deposition. The collection means can be wrapped in an appropriate cellophane or equivalent wrap and can be provided sterile (gamma irradiation or ethylene oxide). Alternatively, a mechanical or electromechanical dispensing device may deliver the stimulant. The dispensing device may also be included as part of the saliva monitoring device of the present invention.

The separate processes useful for separate functions in the construction of a one-step device of the present invention are described below. Four (4) different active processes employing separate principles are described. Various combinations of these processes can be employed based on the materials selected and the specific device designs required to facilitate separation and guarantee delivery of a minimal volume of processed saliva. The sample volume that needs to be collected depends upon the collection principle used (e.g., aspirate vs. gravity) and the design of the collection device. As such anywhere from 2 to 4 of the processes can be utilized at any one time as described below to accomplish the objective. All of the varied combinations described are viable approaches and as such the device design dictates in part the processes that need are used. As such one cannot merely separate the processes from the design as the two together accomplish the function.

Multiple combinations of processes and designs are hence provided and defined hereafter.

The minimal sample volume that typically needs to be delivered to an electrochemical sensor strip is 3 micro liters (μl). Most sensors work best with 5 μl with no upper volume restraint. Any saliva collection device will need to reliably deliver a minimal volume of processed saliva (approx. 5 μl). The amount of stimulated saliva that needs to be collected to deliver the minimal volume is dependent upon device design and the number and type of processes involved. The materials used in device design may retain sample and the amount retained needs to be accounted for to make minimal sample volume delivery failsafe. As such different combinations of active processes utilizing different principles and different device designs have been engineered to meet the requirements: minimal sample volume delivery; and delivery of fluid relatively free of interfering materials.

Device designs may involve several means for initial (primary) sample fluid collection. These include: expectoration (spitting) of saliva fluid into a container; aspiration of stimulated saliva fluid from under the tongue or other pooled fluid collection site such as the cheek within the mouth; scooping of fluid from under the tongue that has been allowed to pool; spontaneously wicking fluid from the pool under the tongue by either touching or holding the collection material in place for a required period of time. Rapid saliva collection by aspiration, or wicking is required.

As such collection and processing devices can be constructed to be either operator passive or operator interactive. Operator passive procedures include, e.g., scooping, wicking, or the use of gravity. Operator interactive procedures include, e.g., aspirating, application of pressure, or dispensing. Saliva collection and processing devices representing multiple versions of both operator types will be described herein along with the compatible operating processes.

A variety of methods are available to help facilitate saliva fluid movement from processing media to processing media within a collection device. A processing media is defined as a material designed to facilitate a specific process step such as a wick or membrane. Hence each processing media is represented by a suitable material such as a membrane to facilitate that processing step in any given device. Contact and transfer between processing media is obviously critical for both saliva processing and for accurate volume delivery. The operating means described below can be used to facilitate fluid movement from processing media to processing media. These methods can include the use of applied pressure, gravity, head volume pressure (in a collection well), angle or cut of the processing media, shape of media, surface area of contact between media, method of contact between media, method of assembly of media in the device. A variety of these methods can be incorporated into any one device design depending upon the number of processes utilized and the part design.

Suitable media include any material of appropriate construction for the process required. Media can be membranes, molded material, extruded material, or the like including housing design. Any shape necessary to complete the function can be utilized. Fluid may move through the fluid by any means deemed necessary. In the case of membranes, saliva can be forced through the membrane (vertical flow) or along the membrane (horizontal flow) depending upon the need.

Media can be held together to create the device by any means necessary based on the design. This may include, e.g., compression fit, welding, adhesion, ultrasonic welding, heating, stapling, use of adhesives, etc. Media may also be held together using plastic devices. Plastic devices are well known in the art and can be blow molded, thermoform molded, or extruded molded plastic parts. Any number of plastics and resins can be used with the provision that glucose not bind non-specifically. In addition, the device design can include, e.g., the use of one-way valves, living hinges, pipette bulbs, aspirators, pressure valves, release layers, dissolving layers, and various other ergonomic design factors, etc, as required. Alternatively the processing and collection device can be constructed from non-plastic, paperboard materials.

After processing several means can be used to deliver processed saliva to the sensor strip. These may include, e.g., touch to strip (transfer by capillary withdrawal, transfer, or wicking); dispensing onto strip (through pressure dispensing, i.e., squeezing, or gravity); or snap strip into collection device (for transfer by contact). All of these media, media factors, designs, and design factors will be utilized as examples in the 4 operative interactive and 4 operator passive designs described later (See Examples Section). Each design will use a variety of process combinations based on the designs and principles used.

Processes, Combinations, and Integrated Designs of the Invention

The present invention provides for the following active processes and specific combinations thereof can be utilized with the appropriate device design to facilitate saliva collection and processing for monitoring purposes. These active processes utilize different processing media. First, the individual active processes will be described by themselves (as separate processes) in order to define the principles involved for each. Secondly, viable combinations of active processes will be described as the basis for design of a device. Third, specific designs incorporating those active processes and the appropriate methods and processing media will be described in the last section.

Four distinct basis (4) processes are provided by the present invention as defined below. The use of all 4 basic processes is not required for construction of a viable device design. Combinations can consist of 2 to 4 selected processes. Not all combinations are useable and as such non-feasible combinations will not be cited. The four processes are defined below and designated, in order, as process “a”, process “b”, process “c” and process “d”. This order indicates the order for which saliva is to be processed upon initial contact. Hence, if all four processes are used, the sequence for saliva processing is as follows: “a” process goes to “b” process goes to “c” process goes to “d”. Two, three, and four active process combinations and designs are described below in increasing order of complexity. For example, a two step design may use ac (i.e., a two-step design including process a and process c) or bd as processes; a three step design, abc or abd; and a four step design process, abcd. In addition, some processes will be designated as 1 or 2; namely 1 representing 1 variant, and 2 a second variant of that process. For example, the use of a membrane for process “c” on a flow thru (vertical) basis will be designated c1; and on a horizontal basis, c2. Other variants will be described after description of each process.

The processes that can be used for active saliva processing in the present invention are summarized in Table 1 below.

TABLE 1

| Designation | Active Process |
|---|---|
| a | Axial Filtration: Axially Directed Migration of |
| | Aqueous Fluid Containing Analyte with |
| | Differential Partitioning of Insoluble Particulate, |
| | Gel-like Material, Macromolecules and Soluble |
| | Polymers through Axial Filtration |
| b | Differential Molecular Nanofiltration: Differential |
| | Nanofiltration of Soluble Globular |
| | Macromolecular Components above 2 nm size |
| c | Protein Removal: Protein Based Binding of |
| | Remaining Soluble Interfering Material, with or |
| | without further Chromatographic Separation |
| d | Absorption: Absorption of Glucose at the |
| | Molecular Level with Unimpeded Transit to the |
| | Point of Delivery |

Axial Dispersion and Partitioning (Process “a”)

After stimulation, the first media to be brought in contact with saliva is very tightly bonded, axially aligned, water impermeable cellophane sleeve wrapped, continuous fibers of cross-linked hydrophilic plastic or cellulosic media in cylindrical or rectangular rod stock form (see paragraph below). Contact with this material results in instantaneous axially directed migration of aqueous fluid containing analyte away from the site of initial fluid contact. Any insoluble particulate, gel-like material, globular macromolecules or soluble polymers (like DNA) is instantaneously entrapped by axial filtration along the depth of the filter. This initial process and media allows the selective and preferential transport of aqueous fluid containing glucose away from the point of initial contact and collection coupled with the differential filtration of gross contaminating material.

By analogy, this axially aligned material has a structure similar to a very, very tightly packed cigarette filter encased on the outside in a water impermeable cellophane sleeve. As such aqueous fluid containing soluble analyte rapidly travels axially away from the point of initial contact, unidirectionally to the next media, traveling rapidly along the cross-linked axial lines of the fiber bundles in the media. Due to the very tight bundling and cross-linking of fibers along with the outer wrapping of the rod stock material in a fluid impervious cellophane wrapper, cross-linked mucus (as gel), host cells, gross lysed cellular debris, and microbes as particulate, and DNA (as a long linear polymer tangled polymer) are unable to enter the matrix or migrate axially through it at the same speed, rate or distance as the aqueous solvent front containing the low MW analyte to be measured. Hence, insoluble particulate, gel-like, and large macromolecular material, in addition to soluble polymer-like material remain entrapped at the initial point of sample contact and collection by differential axial filtration. As such, the first active process “a” accomplishes several active functions: rapid axially directed migration of aqueous liquid within the sample; preferential and selective partitioning of the low MW analyte into the rapidly migrating aqueous front based on its soluble nature and small size and low MW (glucose MW 180 Daltons); preferential retention and entrapment of interfering materials at the point of contact; initial partitioning (processing) of the sample; and rapid transit of reactive fluid to the next media and active process. It is advantageous to increase the surface area of the axially aligned material through use of a diagonal cut at the point of initial contact to increase the surface area, amount of entrapment, relative amount and speed of aqueous fluid processed. This may also be beneficial at the point of contact of process a material with the next media.

Continuous micro-fibers of polyester, polypropylene, cellulose acetate, polyolefin, or nylon can be high-speed extrusion bonded into virtually any profile shape. Bonded fiber media is tightly packed and axially aligned (similar in design to a cigarette filter but hydrophilic). As example Filtrona (Richmond, Va.) provides Transorb R XPE bonded filters in 4.0-18.0 mm diameter. Filtrona also provides Transorb R wicks for use in axial flow. These tightly bonded fibers can be impregnated with citric acid as a granular powder or as a liquid additive and then dried to aid stimulation. These bonded fibers can be plastic coated or film wrapped. Aqueous solvent dispersion and partitioning is literally instantaneous along the axially aligned capillaries as the aqueous solvent front rapidly migrates with solute (analyte).

Differential Molecular Nanofiltration (Process “b”)

Following Process “a”, soluble mid-sized globular materials above 2 nm size (the smallest virus is 18 nm) are removed from saliva by passage through nanofiltration membranes wherein materials with a size >2 nm are differentially retained. Nanopore membranes are 180 degrees different from conventional filter membranes, and are only available recently at such low pore sizes based on nanotechnology advances. Nano; indicates 1×E10-9 in size vs micro- which means 1×10⁻⁸ in size, a thousand fold or three orders of magnitude smaller. Nanopore membrane porosity is strictly controlled as discrete highly uniform, circular pores (buckshot like discrete holes) in the membrane similar to what seen in a sieve but only at the molecular level. The membranes are available either in inert hydrophilic plastic or inert hydrophilic alumina silicate or inert hydrophilic ceramic form. All of these membrane types are characterized by their very high hydrophilicity, very high hole density, very thin, and very high flow rates in spite of the small pore size. These membranes are to be differentiated from conventional membranes, which exhibit the opposite features and are constructed in a totally different manner.

Alumina silicate membranes have a hollow tunnel pore structure and are more rigid as they are made of silica. Nanopore membranes have holes in the very low nanometer range whereas conventional filters operate only in the micron (micrometer) range. And, as such, nanopore membranes exhibit extremely high flow rates even compared to larger pore size conventional cross-fiber layered mesh membranes. Nanomembranes remove soluble globular materials at the molecular range of small viruses. Conventional membranes cannot be used for the nanofiltration of samples. Nanopore membranes have a very thin membrane thickness. Typical nanopore membrane pore sizes are as low as 0.01 μm (10 nm) with up to 1×E11 pores/cm² and a flow rate of 0.1 ml/min/cm². For a larger 0.1 μm (100 nm) nanopore membrane, there would be 4×E8 pores/cm², with a flow rate of 2 ml/min/cm². Flow rates for saliva can be somewhat less based on saliva viscosity if not prefiltered properly to remove gross material. Since the membranes are composed of discrete holes there is little resistance to fluid flow through either a gravity or pressure basis.

The nanopore membrane properties unique for saliva use include: nano-pore size level of filtration; highly hydrophilic; non-clogging; thin; and able to withstand pressure or vacuum. As concerns active processes the recent advent of these membranes provides the only technical means to selectively remove insoluble or soluble materials from samples in the range from 2 nm to several hundred million nm in a rapid fashion (<30 sec). The other approaches that work with some precision in the nano-range are very slow and centrifugation (12 hrs at 100,000 g in an ultracentrifuge) is an example.

Nanofiltration of a saliva sample with a 2 nm nanofilter would leave it in a state wherein it only contains soluble protein-like material below 1,500 kDa; 20 nm, 15,000 Daltons. Suitable hydrophilic nanopore membranes are available in the 2-200 nm size or above include ion track-etched polycarbonate membranes (Osmonics, Minnetonka, Minn.), Anopore Inorganic Aluminum Oxide Membranes (SPI, Westchester, Pa.), SPI-Pore Polycarbonate Membranes, and/or Steriltech ceramic disc membranes (Steriltech Corporation, Kent, Wash., and/or any custom nanofabricated, uniform morphology, self-organized, anodic alumina nanodevice arrays constructed for thin film separation purposes.

Protein Binding with Chromatographic Separation (Process “c”)

Following Process b, nanofiltered saliva fluid contains soluble saliva materials with a size less than 2 nm diameter. Most soluble saliva materials with a MW less than 1,500 kDa will be included in this nanofiltrate. The majority of the soluble materials cited in this MW range that are found in saliva are “protein” in nature and include mucin 1 (1,000 kDa), slgA (600 kDa), mucin 2 (150 kDa), IgG (140 kDa), lactoferrin (90 kDa), peroxidases (85 kDa), amylases 980 kDa), carbonic anhydrase (70 kDa), proline rich proteins (50 kDa), lysozyme (20 kDa), statherins (7 kDa), and histatins (3 kDa). slgA has already been removed in the last step.

In order to allow glucose to pass unrestrained in the aqueous phase to the next phase, the sample is processed further to remove soluble, protein-based contaminants between 3 kDa and 1,000 kDa (or any proteinaceous material for that matter). To accomplish this a hydrophilic, high protein binding blotting membrane is used to instantaneously bind all protein materials. Suitable high protein binding membranes (having a binding up to 448 ug/cm2 upon a single pass through) include Immobilon-PSQ polyvinylidene fluoride (PVDF) 0.2 um or larger pore size (Millipore), Prima 40 large pore size direct cast nitrocellulose (S&S) with a flow rate of 10 sec/cm, Porablot NCP PVDF membranes (Machery-Nagel, GE), or the like.

Nanofiltrates (from process b) are allowed to either vertically flow thru the high protein binding membranes (designated c1 for vertical flow-thru) or are applied to one end of a horizontal strip (designated c2 for horizontal flow). Irreversible binding of proteins or protein-containing material is instantaneous upon contact and the protein will remain immobilized at the point of contact allowing the aqueous solvent front to flow unimpeded either horizontally or vertically. In the latter case protein interfering materials are bound to the front edge of the strip and the aqueous fluid containing glucose is allowed to chromatograph down the strip also resulting in the active separation of soluble protein containing materials from glucose in aqueous solvent.

For saliva applications other than glucose detection (which requires the removal of interfering protein containing soluble material), other membranes are available for use to allow just the chromatographic separation of analyte (say a DOA, or TDM test) from unrelated slower migrating species. Membranes with these properties would be useful for analytes in saliva like, e.g., cocaine, amphetamine, methamphetamine, THC, phenylcyclidine, opiates like heroin, steroids like cortisol, aldosterone, testosterone, progesterone, DHEA-S, thyroid hormones like fT4, fT3, therapeutic drugs like cyclosporine, theophylline, Ritalin, psychiatric drugs and the like (as non-inclusive example). Numerous chromatographic paper media have been developed that would allow chromatographic separation of aqueous fluid without removal of proteins yet facilitate a chromatographic separation based on the differential rate of speed of soluble material (slow) from small MW analyte contained in the solvent front (fast). These membranes employ the principle of rapid solvent front (containing analyte) migration ahead of the bulk of denser solution as a result of interaction with the solid phase. The result is partitioning within a sample in either a vertical or horizontal plane in the chromatographic media. This is the principle behind thin layer plate or paper chromatography in a 2-dimensional plane or elution in void volume for 3-dimensional chromatographic separations and can be applied as a principle in saliva separations as well. It is applied here in the simplest sense to facilitate aqueous solvent separation containing dissolved solute (glucose or other low MW analyte) from non-chromatographic materials. Although useful, it is not necessarily preferred as it separates based on chromatography alone, it is cited here as an option. For example, Whatman, and Schleicher and Schuell both offer various macroporous chromatographic separation media that can be selected empirically for specific desired chromatographic migration rates and chromatographic separation properties for use on either a horizontal or vertical flow basis with application to saliva. Selected properties that are useful include speed of flow, wicking speed, separation rate, etc. For example, useful materials include Whatman multi-media composite microfibre membranes such as grades 934-AH, or Multigrade GMF with linear or radial wicking times of 50 sec/7.5 cm at 1 μm; or S&S grade GF10, 53, etc. The above materials would facilitate solvent separation and subsequent chromatographic separation under non-pressure conditions.

Molecular Absorption with Transit (Process “d”)

Following earlier saliva processes, the molecular adsorption and vertical (d1) or horizontal (d2) transit of sample can be employed for final delivery of the conformational correct isoform of glucose or other low MW analyte of choice to a sensor strip or other detection means. As such, glucose as a molecule has an inherent molecular instability of the molecule itself owing to either isomerization or other intramolecular variations. Glucose exists in a left and right form (e.g., D-glucose and L-glucose), the ratio of which can vary spontaneously. Glucose also converts depending upon sample pH and ionic strength to other isomeric forms such as fucose and mannose. Glucose also changes structural form based on rotation around anomeric carbon 2. Hence the reason for inclusion of this step, although optional, can be for the molecular (chemical) separation of selected isoforms of the analyte, such as glucose from fucose, or to facilitate isoform stability. In the case of applications other than glucose, i.e., aldosterone, there can be up to 15 different related steroid species that one may need to select from on a molecular basis. Molecular absorption based on the use of discrete molecular size can be used. This active molecular process “d” constitutes the differential molecular separation of closely related molecular species based on the principle of selective absorption. Both the selection of absorbent and the designed method of use of said absorbent(s) allows these materials to be used in a manner that not only readily and spontaneously absorbs the selected species but also allows the ready transit of aqueous solute containing the analyte through the pore structure to the final point of delivery in a manner which is unimpeded and does not require elution or ion exchange. The materials simply “pass through”.

To facilitate such at the molecular level in the case of glucose (˜180 Daltons), a variety of absorptive materials are available of controlled pore size to allow glucose to enter and pass unhindered through the absorptive matrix. This allows for final separation of glucose from salivary materials at the molecular level. Absorbents can be employed in various designed formats including pressed cakes, pills, column packings, layers between membranes, or for horizontal flow attached to an inert mylar base through a double stick adhesive to allow horizontal flow. Porous absorbents are readily available with intraparticle pore sizes around 300 MW (preferred) for glucose entry and internal surface areas up to 700 M2/gm. Such absorbents include: molecular sieve ABSCENTS and MOLSIV GMP brand of synthetic or natural zeolite based deodorizing powders of highly controlled pore size with internal pore sizes up to 700 M2/tablespoon (UOP International, Des Plaines, Ill.), Versal synthetic aluminum oxide microspheres A 1203, A-201, and A-2 as absorbents (UOP International); synthetic ceramic microspheres as inert absorbents, Zeospheres brand (Lawrence Industries, Ltd, UK), ASP Series hydrous alumina silicate microspheres as absorbents (Lawrence Industries); Dryocel alumina desiccant beads with high surface area (up to 400 M2/gm internal surface area) as high capacity absorbents (Lawrence Industries); Pharmasorb attapulgus clay with high absorptivity at select pore size (Lawrence Industries); Trockenperlon beaded silica gel dessicants as absorbents (Lawrence Industries); natural clay absorbents such as chabazite mineral zeolite ZS500H, ZS500a, ZS500RW, ZS500AA, or A or the like (GSA Resources, Inc., Tucson, Ariz.); additional natural clay absorbents such as: clay Ferrierite CP914; ZSM-5 Type Zeolite CBV 3024E, 5534G, 8014, or 28014; Zeolite Y Type CBV100-901; Mordenite type CBV 10A, 21A, or 90A (Zeolyst International, Valley forge, Pa.); or activated carbon as absorbent under pressure or vacuum (numerous sizes and sources too numerous to list here; available from Nordit, Shundler, Cameron, etc).

Useful Process Combinations of the Device of the Invention

A variety of combinations (up to four of the aforementioned processes) are provided below as viable options for saliva processing for glucose monitoring. Combinations are first listed, followed by specific design considerations thereafter.

Two Process Combinations

In one embodiment of the invention, the glucose monitoring device uses two-process combinations. Two-process combinations useful in the glucose monitoring device of the invention include, but are not limited to, e.g., ac1, ac2, ad1, ad2, cd1, bd1, and bd2.

Three Process Combinations

In one embodiment of the invention, the glucose monitoring device uses three-process combinations. Three-process combinations useful in the glucose monitoring device of the invention include, but are not limited to, e.g., abc1, abc2, abd1, and abd2.

Four Process Combinations

In one embodiment of the invention, the glucose monitoring device uses four-process combinations. Four-process combinations useful in the glucose monitoring device of the invention include, but are not limited to, e.g., abc1d1, abc1d2, and abc2d2.

Designs of the Device of the Invention

Saliva collection and processing devices of the invention can be either relatively operator passive (other than to scoop, allow to wick, or to use gravity) or operator interactive (wherein operator has to physically aspirate, apply pressure, or dispense) in design. Both design types will are useful in the method of measuring glucose and are considered along with different combinations of active processes as noted below.

Operator Interactive Designs

Design 1

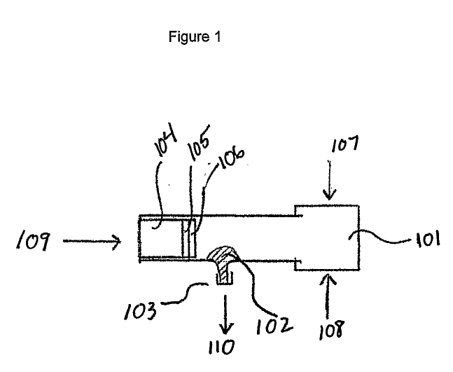

One embodiment of the device of the invention is shown in FIG. 1. Features of the device include, e.g., a squeeze bulb aspirate, a vacuum process, a gravity collect, and a touch delivery. As shown in FIG. 1, in one embodiment of the device of the invention, the device includes a process combination; a squeeze bulb (1); and a removal cap (3). Saliva fluid (2) is also shown in the Figure. In one embodiment of the device of the invention, at least one component of the device is formed of extruded molded plastic. Process combinations useful in the device of the invention as detailed in FIG. 1, include, but are not limited to, e.g., ac1; ad1; c1d1; bd1; abc1, abd1; abc1d1 and abc1 (shown in FIG. 1).

Design 2

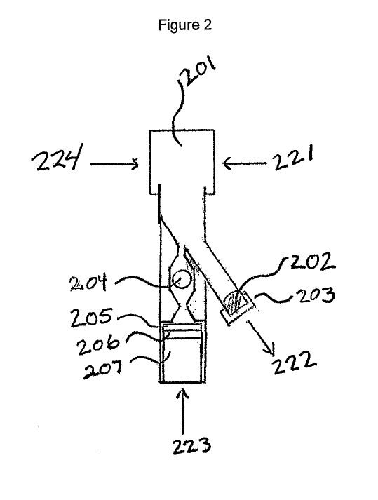

One embodiment of the device of the invention is shown in FIG. 2. Features of the device include, e.g., a squeeze bulb aspirate, vacuum process, and bulb dispense with one-way valve. As shown in FIG. 2, in one embodiment of the device of the invention, the device includes a process combination; a squeeze bulb (1); removal cap (3); and floating ball in one-way valve (4). Saliva fluid (2) is also shown in the Figure. In one embodiment of the device of the invention, at least one component of the device is formed of extruded molded plastic. Process combinations useful in the device of the invention as detailed in FIG. 2, include, but are not limited to, e.g., ac1, ad1, c1d1, bd1, abc1, abd1, abc1d1 and abc1 (shown in FIG. 2).

Design 3

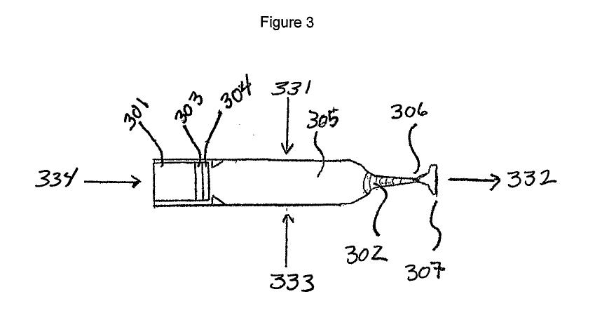

One embodiment of the device of the invention is shown in FIG. 3. Features of the device include, e.g., squeeze barrel aspirate, vacuum process, invert, twist-off cap, and dispense. As shown in FIG. 3, in one embodiment of the device of the invention, the device includes a process combination; squeeze barrel design (5); sealed tip junction, until cap removed (6); and twist-off disposable tip to allow dispensing upon inversion (7). Saliva fluid (2) is also shown in the Figure. In one embodiment of the device of the invention, at least one component of the device is formed of blow-molded plastic. Process combinations useful in the device of the invention as detailed in FIG. 3, include, but are not limited to, e.g., ac1; ad1; c1d1; bd1; abc1; abd1; abc1d1; and abc1 (shown in FIG. 3).

Design 4

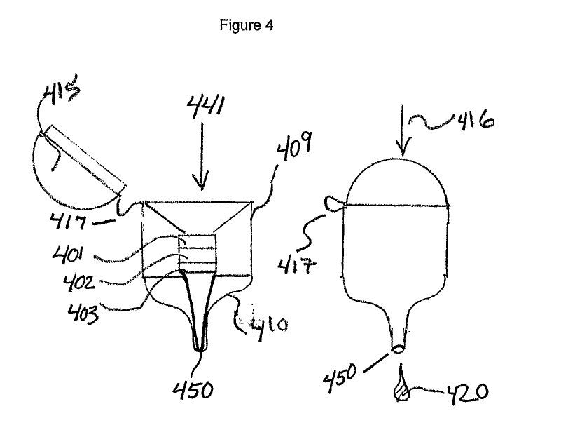

One embodiment of the device of the invention is shown in FIG. 4. Features of the device include, e.g., collect expectorate in cavity, snap cap into place (attached via living hinge), hold upright and squeeze, pressure process, and dispense. As shown in FIG. 4, panel A, in one embodiment of the device of the invention, the device includes a process combination; open squeeze top (15); top housing (9); bottom housing (10) As shown in FIG. 4, panel B, in one embodiment of the device of the invention the device includes a closed squeeze top (16); a living hinge (17). Saliva fluid (2) is also shown in the Figure. In one embodiment of the device of the invention, at least one component of the device is formed of blow-molded plastic or extrusion-molded plastic, or combination thereof. Process combinations useful in the device of the invention as detailed in FIG. 4, include, but are not limited to, e.g., ac1, ad1, c1d1, bd1, abc1, abd1, abc1d1 and abc1 (shown in FIG. 4).

Operator Passive Designs

Design 5

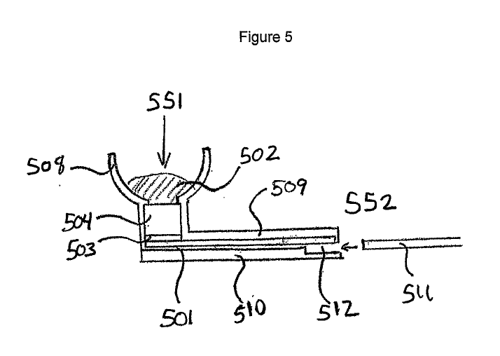

One embodiment of the device of the invention is shown in FIG. 5. Features of the device include, e.g., collect expectorate, gravity process, and touch or snap to dispense. As shown in FIG. 5, in one embodiment of the device of the invention, the device includes a process combination, well to expectorate sample into (8); top housing (9); bottom housing (10); sensor strip for insertion (opening) (12); sensor strip (11). Saliva fluid (2) is also shown in the figure. In one embodiment of the device of the invention, at least one component of the device is formed of molded plastic. Process combinations useful in the device of the invention as detailed in FIG. 5, include, but are not limited to, e.g., ac2; ad2; c1d2; bd2; abc2; abd2; abc1d2; and abc2 (shown in FIG. 5).

Design 6

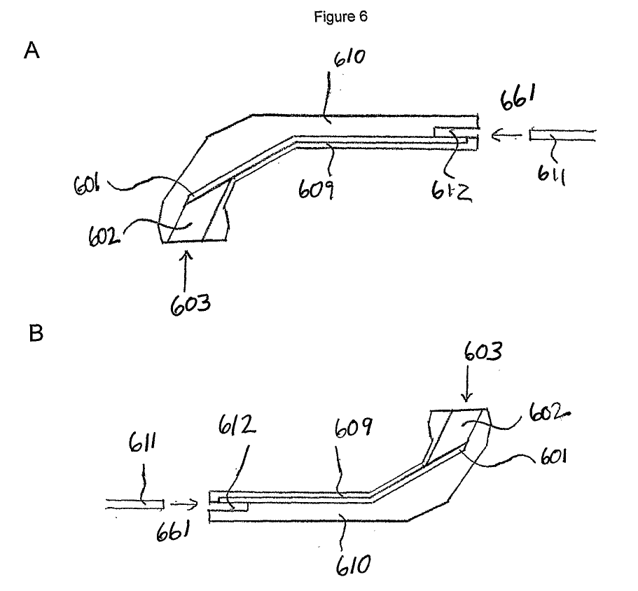

One embodiment of the device of the invention is shown in FIG. 6. Features of the device include, e.g., angled wick collect from under tongue, flip over, gravity/angle process, and touch to sensor or snap in sensor to dispense. FIG. 6, panel A shows the aspirating model of the embodiment of the device of the invention. FIG. 6, panel B shows the running mode of the embodiment of the device of the invention. As shown in FIG. 6, panel A and panel B, in one embodiment of the device of the invention, the device includes a process combination; a bottom housing (10); a top housing (9); sensor strip point of insertion (opening) (12) and sensor strip (to be inserted) (11). In one embodiment of the device of the invention, at least one component of the device is formed of molded plastic. Process combinations useful in the device of the invention as detailed in FIG. 6, include, but are not limited to, e.g., ac2; ad2; abc2; abd2; abc1d2; and ac2 (shown in FIG. 6).

Design 7

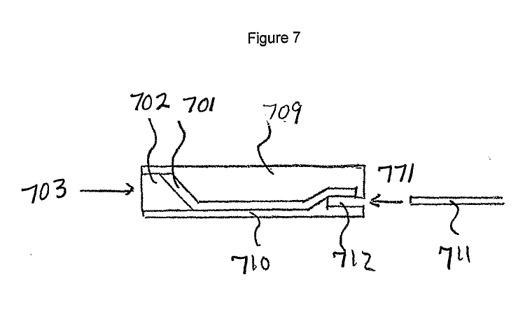

One embodiment of the device of the invention is shown in FIG. 7. Features of the device include, e.g., straight wick collect, invert and hold 1 minute, gravity process, touch or snap to dispense. As shown in FIG. 7, in one embodiment of the device of the invention, the device includes a process combination; top housing (9); bottom housing (10); sensor strip point of insertion (opening) (12); and sensor strip (to be inserted) (11). In one embodiment of the device of the invention, at least one component of the device is formed of molded plastic. Process combinations useful in the device of the invention as detailed in FIG. 7, include, but are not limited to, e.g., ac2; ad2; abc2; abd2; abc1d2; and ac2 (shown in FIG. 7).

Design 8

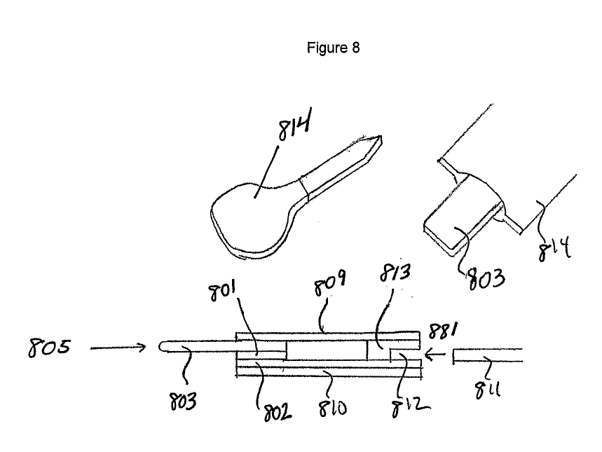

One embodiment of the device of the invention is shown in FIG. 8. Feature of the device include, e.g., touch wick collect or hold in mouth, gravity/chromatographic process, touch or press or snap to dispense. As shown in FIG. 8, in one embodiment of the device of the invention, the device includes a process combination, paper housing (14); top housing (9); bottom housing (10); sensor strip point of insertion (opening) (12). In one embodiment of the device of the invention, at least one component of the device is formed of molded plastic or paper. Process combinations useful in the device of the invention as detailed in FIG. 8, include, but are not limited to, e.g., ac2; ad2; abc2; abd2; abc1d2; and abc2 (shown in FIG. 8).

### EXAMPLES

### General Information Relating to Example 1 to Example 5

Materials and Methods

Each of the 4 active processes defined earlier was studied for suitability for glucose processing. Various representative media (membranes, filters, papers, materials) with manufacturer pre-established verified specifications for the membrane properties for which they are inherently designed, validated, and used for (as example: 1) axially directed migration and filtration of aqueous fluid by axial filtration; 2) differential nanofiltration of soluble macromolecular components; 3.) protein based binding of remaining soluble protein material; and 4.) absorption of glucose at the molecular level) were obtained from commercial sources and tested. All studies were carried out in two stages: 1.) rule out glucose binding to the media or contribution of glucose from the media so as to interfere with glucose test results and use of the media for its manufacturer's stated use; and 2.) examination of suitability of use of the material as a media for stimulated saliva processing from patients for glucose measurement). In these studies it was not the intent nor was it necessary to re-demonstrate manufacturer established claims for the various specialized purposes for which the media were constructed (e.g., protein binding to nitrocellulose, or nanofiltration of macromolecules) as the usage of these materials for these purposes has been fully established by each manufacturer. These materials are in routine use for these purposes for various other applications. The focus here was in clinical validation and utilization of these media for glucose processing from saliva relative to reference methods and that constituted validation of the described process.

### Example 1

### Process a

Rule Out Glucose Binding Over Time to Axial Dispersion Wicks.

Transorb™ Wicks type R-22596 of 4.75 mm diameter composed of bonded polyolefin were obtained from Filtrona Richmond, Inc., Richmond, Va. To rule out glucose binding, fifty (50) ml of a standard glucose solution at a 5 mg/dl concentration in distilled water was placed in a polystyrene Petri dish and a 6 cm long wick was allowed to set in the solution on end for approximately 30 seconds until liquid moved up the wick. After filling, each wick was allowed to incubate for 5 min., 30 min., or 60 minutes after before further processing. Each time point comprised 3 separate wicks as replicates (n=3). After incubation, each of the three wicks per time point were hand extruded by pressing from the side that touched the liquid to the end that did not touch the liquid by inverting the wick over a test tube and pressing. The first drop of extruded fluid that had transversed the wick was tested for glucose for recovery. Recovery constituted no binding to the media even after prolonged incubation. In clinical practice wicks are processed within 1 minute of collection of saliva.

Testing for glucose was done on a Yellow Springs International (of SI) 2700 Auto-analyzer SELECT, which uses a reusable platinum electrode, and glucose oxidase coated membranes (YSI Glucose Membranes YSI 2365) for the amperometric detection of glucose. An aliquot of the standard glucose solution was obtained from the petri dish prior to wick addition as control (100% recovery) and wicks immersed in distilled water without glucose were also run as controls.

Calibration of the YSI 2700 for glucose measurement was done daily prior to testing as per manufacturer instructions. AYSI glucose standard at 500 mg/dL was prepared in YSI Buffer (YSI 2357 Buffer Concentrate). Calibrators at various concentrations were prepared from the YSI standard by dilution of the standard in distilled water. Calibrators covered the range from zero to 20 mg/dL in 0.5 mg/dL increments. Calibrators were run in duplicate by a CLS technician several times daily using a 65 microliter sample size and a 15 second reading interval. Results were automatically recorded by the instrument and expressed in both nano amps and mg/dL.

Results are shown in Table 2.

TABLE 2
Binding of Glucose to Axial Filters

| | Sample | | | Percent | |
|---|---|---|---|---|---|
| Sample | Current | Concentration | | (%) | Percent (%) |
| Tested | (nA) | (mg/dl) | Mean | Recovery | Contribution |
| Control *1 | 0.64 | 4.77 | | | |
| Control *2 | 0.65 | 4.82 | | | |
| Control *3 | 0.65 | 4.78 | 4.8 | 00% | |
| 5 min 1 | 0.78 | 5.78 | | 121 | |
| 5 min 2 | 0.73 | 5.43 | | 113 | |
| 5 min 3 | 0.74 | 5.44 | | 113 | |
| 30 min 1 | 0.77 | 5.86 | | 118 | |
| 30 min 2 | 0.81 | 5.93 | | 122 | |
| 30 min 3 | 0.7 | 5.08 | | 106 | |
| 60 min 1 | 0.71 | 5.13 | | 107 | |
| 60 min 2 | 0.72 | 5.14 | | 107 | |
| 60 min 3 | 0.8 | 5.69 | | 119 | |
| Control **4 | 0 | 0 | | | 0 |
| Control **5 | 0 | 0 | | | 0 |
| Control **6 | 0 | 0 | | | 0 |

*No Wick
**Wick with water (no glucose)

As shown in Table 2, the average percent recovery over 60 minutes exceeds 100% indicating glucose was not absorbed to the fibers of the Transorb™ Wicks. A marginal increase in glucose concentration was observed due to rehydration of the wick material itself resulting in a slight increase in recovery of glucose but no binding of glucose was observed, nor do Transorb filters contribute glucose.

### Example 2

### Process b

Rule Out Glucose Binding to Molecular Nanofilters.

SPI-PORE™ Standard White Polycarbonate Track Etch Screen Membrane Filters, #E 5013 (13 mm diameter; 0.01 micrometer (10 nm) pore size) and AnoPore™ Inorganic Aluminum Oxide Membrane Filters (13 mm diameter; 0.02 micrometer (20 nm) pore size) were obtained from SPI, Westchester, Pa. Standard stainless steel filter holds were also obtained to hold the membranes and provide the means to add glucose solution through use of a syringe and a dedicated port.

Filters were assembled in holders and either a 0, 0.5, or 1.0 mg/dL solution of glucose in distilled water was allowed to pass through each filter type by first drawing the glucose standard solution into a 1 cc syringe, attaching the syringe to the filter assemble by luer-lock, and gently pushing the liquid through the filter using light pressure. The glucose concentration was determined before and after filtration. Unfiltered material represented 100% recovery.

Results are shown in Table 3.

TABLE 3
Binding of Glucose to Nanofilters
Amount recovered*

| | Glucose | Sample | | | |
|---|---|---|---|---|---|
| | Added | Current | Conc. | % | % |
| Membrane | (mg/dL) | (nA) | (mg/dl) | Rec | Contribution |
| None | 0 | 0 | 0 | | |
| | 0.5 | 0.11 | 0.451 | | |
| | 1 | 0.22 | 0.947 | | |
| Polycarbonate | 0 | 0 | 0 | | 0% |
| | 0.5 | 0.11 | 0.472 | 100% | |
| | 1 | 0.23 | 1.091 | 110% | |
| Aluminum Oxide | 0 | 0 | 0 | | 0% |
| | 0.5 | 0.11 | 0.467 | 100% | |
| | 1 | 0.22 | 0.96 | 100% | |

*Mean of 3 relplicates

As shown in Table 3, polycarbonate or aluminum oxide molecule membranes under standard use did not retain glucose. Neither membrane contained glucose, which was detected in the YSI 2700.

### Example 3

### Process c

Rule Out Glucose Binding to Nitrocellulose.

The same setup as used in Example 2 was used for nitrocellulose membranes. The only difference was nitrocellulose membrane was used. Prima 40 direct-cast nitrocellulose with a flow rate of 10 sec/cm and a pore size of 1.0 micron was obtained from Schleicher and Schuell, Keene, N.H.

Results are noted in Table 4. Glucose binding to or glucose contribution from the membrane was not observed under the conditions of membrane use.

TABLE 4
Binding of Glucose to Nitrocellulose

| | | Sample | | | |
|---|---|---|---|---|---|
| | Analyte | Current | Conc | % | % |
| Membrane | Added (mg/dL) | (nA) | (mg/dL) | Recovery | Contrib |
| None | 0 | 0 | 0 | | |
| | 0.5 | 0.11 | 0.46 | | |
| | 1 | 0.22 | 0.951 | | |
| Nitrocellulose | 0 | 0 | 0 | | 0% |
| | 0.5 | 0.12 | 0.481 | 104 | |
| | 1 | 0.23 | 1.087 | 114 | |

### Example 4

### Process d

Rule Out Glucose Binding to Absorbent

Extended rod stock zeolite crystals type CBV 500 CY1.6 (lot #98-18) was obtained from Zeolyst, Inc. To 0.9 gm of Zeolite in a test tube was added 2 ml of 0 or 1 mg/dL glucose in distilled water. Samples were allowed to incubate at RT for 30 min to allow glucose absorbance. After incubation, excess liquid was thoroughly drained and the Zeolite crystals were washed twice with 2 ml of distilled water. Zeolite samples in tubes were gently vortexed for 60 seconds following addition of 600 ml of 4% KCL to release any absorbed glucose by ion exchange. Samples were run in triplicate and controls included no Zeolite. Results were run in triplicate and controls included no Zeolite.

Results are shown in Table 5, demonstrating that Zeolite absorbs glucose which can be removed by ion exchange (to demonstrate the principle) and Zeolite does not naturally contain glucose as measured by the YSI 2700.

TABLE 5
Binding of Glucose to Zeolite

| | Glucose | Sample | | Conc. | | % |
|---|---|---|---|---|---|---|
| | Added | Current | Conc. | Adjusted* | % | Contribu- |
| Zeolite | (mg/dL) | (n/A) | (mg/dL) | (mg/dL) | Rec | tion |
| CBV 500 | none | 0 | 0 | 0 | | 0 |
| | | 0 | 0.0132 | 0.039 | | 0%** |
| | 1 mg/dL | 0.04 | 0.165* | 0.5 | 47% | |
| | | 0.03 | 0.132* | 0.4 | 37% | |
| | | 0.04 | 0.192* | 0.58 | 54% | |
| None | 1 mg/dL | 0.081 | 0.361 | 1.07 | | |

*The dilution factor was 1 to 3 as 200 ml of zeolite solution was used for absorption and 600 ml of 4% KCL was used for elution. After adjustment for dilution there was on average a 50% recovery of analyte from the Zeolite.
**Negligible (noise)

### Example 5

### Patient Testing

To demonstrate feasibility, a Three Process Combination of media, namely abc1, simulating operator-assisted designs 1, 2, 3 & 4, was tested on patient's samples. The Three Process Combination that was employed for abc1 used Transorb™ Wick type R-22596 (Process a), SPI-PORE™ Standard White Polycarbonate Track Etch Screen Membrane Filter #E 5013 (0.01 micron) (Process b), and Prima 40 direct-cast nitrocellulose (process c.). The procedure employed was as follows: a 5 cm length Transorb™ Wick was used to adsorb saliva at one end; after absorption (˜1 minute), the wick was inverted, fitted with a squeeze bulb, and the fluid in the wick was dispersed from the other end of the wick under pressure following touching the other end of the wick to a 13 mm diameter stack of SPI-PORE Polycarbonate membrane on top of a 13 mm Prima 40 nitrocellulose membrane (held in the filtration fixture) to which a mild vacuum was applied to the opposite side. Saliva processed through the three media was collected and tested in the YSI 2700. The total time from collection to final processing was less than 5 minutes.

The clinical study involved a total of 27 patients of varying age, gender and geographic location. The group consisted of 12 confirmed diabetics, 6 hypoglycemic patients, and 9 normal patients. Finger stick blood glucose values were available on 11 out of the 27 patients, 7 from the diabetic group, 1 hypoglycemic, and 3 normal.

To properly collect samples, patients were advised to take 20 mg citric acid orally to stimulate saliva production. Within 30 seconds the Transorb™ Wick was placed in the pool of fluid under the tongue and allowed to instantly wick the stimulated salivary fluid. The wick was removed from the mouth, fitted with a plastic squeeze bulb and fluid was dispersed onto a stack of polycarbonate membrane on top of nitrocellulose membrane stock under slight vacuum. Processed fluid was tested immediately in the YSI 2700. The YSI 2700 was fully calibrated in advance.

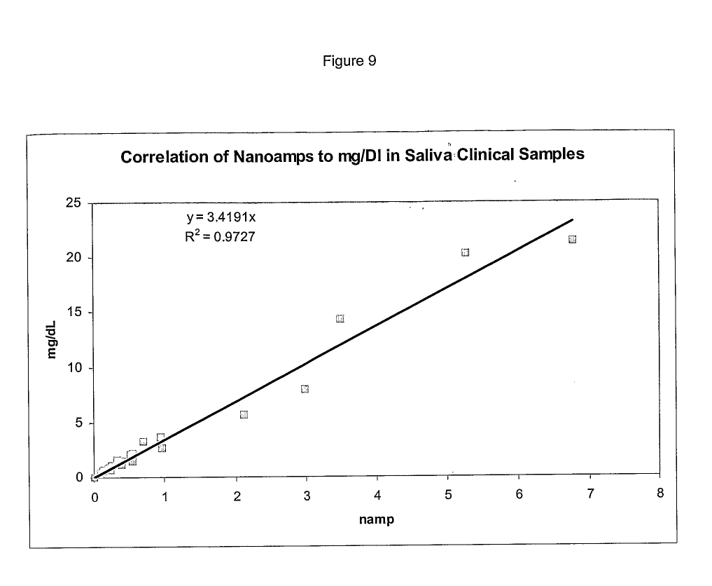

FIG. 9 shows the relationship of nanoamps to mg/dL values in saliva for the patients studied. Processed saliva gave glucose values ranging from 0 to 7 mg/dL in this study group. A linear relationship was observed between current and glucose values with an R2 value of 0.97 indicating an excellent correlation of current to concentration with saliva samples.

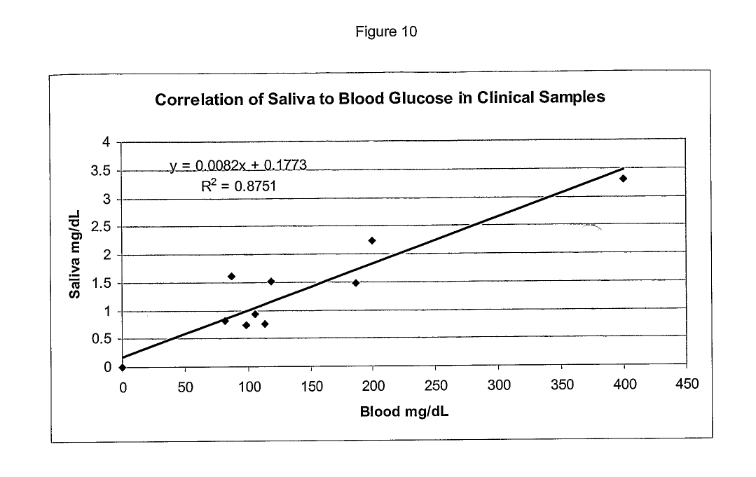

FIG. 10 shows the correlation of stimulated saliva glucose values to finger stick whole blood values collected at the same time. A linear dose response was observed between saliva and blood glucose values as measured in the YSI2700 with an R2 of 0.88 indicating an acceptable correlation. All diabetic patients gave saliva values consistent with blood values. Some variability in individual replicate values was noted in the study attributable to either the small study population, running patients in singlicate only, use of different over-the-counter blood monitors at patient's discretion, and use of improvised processing conditions in lieu of a molded plastic device and squeeze bulb.

### Example 6

### Co-Tracking Clinical Algorithm for Saliva Monitoring

In one aspect, this invention describes a unique clinical algorithm that can be applied to consumer use that allows for the ready transition back and forth between blood and saliva to assure monitoring accuracy between both body fluids at the individual patient level. This algorithm is applicable to a clinical situation wherein either fluid is measured intermittently at will.

Diabetics routinely monitor their blood glucose levels over time. This is the standard practice. Over years of regular tracking of blood values the patient has not only developed the skill and mentality for monitoring but has been able to follow diet guidelines and insulin injections in the case of type 1 diabetes to help manage their condition. Fingerstick whole blood is the diabetic's only choice. Most diabetics have an aversion to taking up to 6 fingersticks a day. This is particularly difficult in the aged or pediatric population. In the elderly eyesight can be a problem and fingers get scarred from repeated use. A reliable alternative to blood is highly desirable. A method that compliments blood testing habits is even more desirable.

Blood monitoring means that patients have also developed a history, whether it be recorded or not, of what their expected blood values are relative to their condition. Now since diabetes is both a progressive disease and a reversible disease (in the case of type 2), it is probable that anticipated values obtained over time are likely to change whether the patient is cognizant of it or not. Drifting in an individual patient's values does occur over time. This would be evident no matter what body fluid is used to measure glucose. As such in some cases it can be important or necessary for patients to track both saliva and blood values over time. The present invention provides a clinical algorithm that can/may be applied to consumer use that aids in the ready transition back and forth between blood and saliva samples when a patient continues to track both body fluids. In order to assure monitoring accuracy at the individual patient level wherein fixed level cutoffs based on population averages do not afford the tracking means over time for accurate monitoring, a unique tracking algorithm based on an individual's unique blood and or saliva baseline values measured over time was developed. This is also important for patient self management as the glucose values reported for blood and saliva are at different concentrations based on the lower level in saliva. Saliva values are approximately 1/50th of those found in blood.

Measurement Parameters of the Invention

Since saliva concentrations are much lower than blood and mean nothing relative to published ADA levels used for blood (8 hr: <110 mg/dl, normal; >=110-<126, impaired; >126, diabetic or 2 hr: <200 mg/dl, normal; >200 diabetic) it will be necessary to express saliva values in “saliva blood equivalents” so that the same reference system (blood) is utilized for reporting. To do this the existing blood algorithm as programmed in the instrument useful for measurement (which must cover the entire glucose dynamic range from 0-800 mg/dL) are reported as saliva blood equivalents as well. As such, this keeps saliva measurements linear with and on the same scale as blood although saliva values are measured in the region from 0 to 25 mg/dl.

Saliva results can be determined as nanoamps and converted to blood equivalents via the embedded mathematics. Two point (or more) blood-based calibration master curves are used as programmed into the master method database software for the instrument. Some instrumentation can use full standardization for calibration curve determination. Either is suitable as required for blood.

An electrochemical sensor technology that affords sensitivity between 0-5 mg/dl, linearity from 0 to 800 mg/dl is used to cover both the saliva and blood dynamic curves within the same preprogrammed calibration run for each lot of product. This allows the use of the same precision offered by blood (to the hundredth decimal point). This also allows for the ability to use the master curve embedded in each lot of product released (as lots of sensor strip and or instrument can be matched and released with a unique master curve for calibration) and the associated master methods database and any methods used to calibrate the blood based meters upon release. Instrument screen flows are modified to allow the option for either/both saliva and blood based testing using the same instrument and sensor strips.

Co-Tracking Methodology

Patient's baselines can vary over time. Patient's metabolism can vary over time. Patient's dietary habits can vary over time. Patient's time of testing can vary over time. Patient's time since last meal (fasting time) can vary daily. The amount of food consumed at the last meal can vary. All of these factors are known to dynamically influence patient blood glucose levels as well as saliva. Based on their metabolic condition diabetic's are however prone to rather habitual patterns of rationed food intake and control, and testing times. Diabetic's are skilled at level loading their glucose intake in spite of the dynamic variables noted above. As such these personal practices and learned routines allow saliva to be used as a surrogate non-invasive fluid for monitoring control when a patient chooses or has the need to measure both fluids at-will over time.

All of these factors are accounted for with the habits diabetics have established for themselves for monitoring their blood level. The is likely a need to be established and monitored for saliva as well. A way to track both in order to come up with a universal algorithm is to establish patterns for saliva and blood through the process of co-tracking over a season of time. The quality of tracking and control can determine the ability to switch between samples at-will and allows the patient to be comfortable with either result. The co-tracking methods described below, coupled with the measurement methods cited above constitute the invention.

Since monitoring levels are patient specific and not population derived, co-tracking is standardized on a per patient basis as the basis for generation of individual tracking algorithms. Clinical studies are conducted prior to release of any product and hence algorithms are developed up front. The approach for the clinical studies to establish the final algorithm is to use actual patient testing values over time as the data for a patient specific individual algorithm tailored to individual patient baseline, diet, medical condition, and testing frequency. The individual algorithm is continuously self adjusting as a rolling average over time that looks at the concordance and deviation in both blood and saliva levels as the basis for steady state monitoring that is panic value risk free.

For the clinical study, testing is as follows for the first eight weeks. For the first 4 weeks of initial use, each patient trains the instrument and generates individual baselines as basis for the individual algorithm. The next 2 weeks is used to confirm the algorithm on a working basis. The last 2 weeks is the saliva solo run. Successful completion of the 8 week co-tracking program allows blood or saliva at-will use. If saliva only testing is chosen at will, periodic blood level checks is continued at weekly and biweekly intervals for type 1 and type 2 diabetics, respectively, to assure baseline consistency between the 2 body fluids.

Individual algorithms are analyzed by conjoint analysis as the basis for the population algorithm to be programmed into the instrument for actual field use. It is likely the population algorithm for type 1 and 2 diabetics are different as they are different disease conditions. The conjoint analysis can determine that. In addition the analysis identifies any necessary covariates that need to be tracked or entered into the final population algorithm for actual field use. As such the clinical data that affords accuracy will define the testing pattern, not the wishes of marketing.

Clinical Study and Analysis for Co-Tracking Methodology

On day zero before testing, patients enter their sex, height, weight, age, type of diabetes (1 or 2), years since diagnosis, number of dental crowns, number of bridges, history of xerostima, smoking status, eyesight status (+−diabetic retinopathy), numbness in extremities, amputations, into the personal monitor as prompted by the screen on the monitor

Testing for week one constitutes blood sample testing only, 6 times a day as follows: upon rising, mid morning or 2 hrs after breakfast, immediately before lunch, mid afternoon or two hrs min after lunch, before dinner, and in the evening 2 hrs after dinner. The time of each meal, the relative caloric intake per meal, and the time of testing is recorded in the monitor as well

The second and third weeks involve the same routine but blood and saliva are both tested

The fourth week involves saliva alone with once daily blood values upon rising.

The “Set Program Algorithm” option is then chosen and the instrument calculates the individual algorithm

The fifth and 6th weeks involve saliva testing 6 times daily and blood once per day for type 1 diabetics and once per 2 days for type 2 diabetics; this confirms the algorithm or fine tunes it further if required.

If the testing values for the 5th and 6th weeks fall within the baseline deviation guidelines, the patient is allowed to test saliva only thereafter.

Analysis Methodology

Depending upon the severity of the disease, one of two different methods is used to determine the individual tracking algorithm specific to the patient. Type 2 diabetic calculations made by the instrument follow guidelines similar to Levy-Jennings criteria for tracking calibrators as follows. Rolling mean blood values are determined along with the standard deviation (SD) and percent coefficient of variation (% CV). A deviation from the saliva baseline mean sufficient to signal blood testing are >+−1 SD (i.e., one (1) standard deviation) from the rolling mean obtained twice in a row in one day. These criteria are useful for saliva provided the second and third week of initial tracking show the precision in both blood and saliva is +−7.5% or less between the 6 daily runs and +−10% or less between daily runs for 14 days running. Panic values warranting contact of the health care provider or doctor are >+/−2SD obtained one time in a row.

Type 1 diabetic calculations made by the instrument follow stricter guidelines owing to the need for insulin injection. Rolling mean blood values are determined along with standard deviation (SD) and percent coefficient of variation (% CV) as before. A deviation from the saliva baseline mean sufficient to signal testing are >+−5.0% from the rolling mean obtained twice in a row in one day. These criteria are used for saliva provided the second and third week of initial tracking show the precision in both blood and saliva to be +−5.0% or less between the 6 daily runs and +−7.5% or less between daily runs for 14 days running. In addition these percentages can be adjusted up or down based on the covariates or disease sequalae noted below. Panic values warranting contact of the health care provider or doctor are >+−1.0-1.5 SD obtained one time in a row.

Type 1 diabetic values (% dev from the mean) considered deviant from the rolling mean are raised or lowered based on certain covariate criteria or disease sequalae as follows:

Deviation from mean value limit of 7.5% raised (raised categories are not additive):

| Caloric intake <800 cal/meal w/in 2 hrs | no increase |
|---|---|
| Caloric intake >800 cal/meal to | +2.5% |
| <1600 cal/meal w/in last 2-4 hrs | |
| Caloric intake >1600 cal/meal to | +5% |
| <3200 cal/meal w/in last 2-4 hrs | |
| Body mass index > 15% | +1.5% |
| Body mass index > 30% | +3.0% |
| Two bridges | +1.0% |
| Smoker | +1.75% |
| Two bridges plus smoker | +2.5% |

Deviation from mean value limit of 7.5% lowered (lowered categories are not additive):

| Numbness | no increase |
|---|---|
| Diabetic retinopathy | −1% |
| Amputation | −2% |
| Retinopathy and amputation | −3% |

Raised or lowered criteria are however additive if factors from both separate categories are present. As example, a type 1 smoker with two bridges, with retinopathy and an amputation would be +−7.0% (7.5%+2.5%−3%). A smoker with a BMI of >30%, with diabetic retinopathy would be +−11.0% (7.5%+3.0% for BMI+1.5% for smoker−1% for blindness). Caloric intake would add to this.

The clinical study generates numerous individual algorithms. These are analyzed by conjoint analysis as the basis for population based algorithms. The population based algorithms programmed in to the instrument for field use can vary dependent upon the covariables identified in the clinical study as contributing to patient result outcome. An option can be provided that criteria may change as warranted by the patient's medical condition or a physician's input.

### EQUIVALENTS

While the invention has been described in connection with the specific embodiments thereof, it will be understood that it is capable of further modification. Furthermore, this application is intended to cover any variations, uses, or adaptations of the invention, including such departures from the present disclosure as come within known or customary practice in the art to which the invention pertains.

1: A method of determining salivary glucose levels in a mammal comprising: obtaining a sample of saliva from the mammal, processing the sample thereby substantially purifying the saliva, and analyzing the processed sample for the presence of soluble carbohydrates, wherein a quantity of salivary carbohydrates in the processed sample correlates with blood carbohydrate levels in the mammal.

2: The method of claim 1, wherein processing the sample further comprises filtering the sample to partition low molecular weight analytes from high molecular weight contaminants and particulate matter.

3: The method of claim 2, wherein filtration is accomplished through axially directed migration of the sample through tightly packed axially aligned fibers.

4: The method of claim 2, wherein filtration is accomplished through one or more nanopore membranes, the nanopore membranes having a median pore diameter from about 200 nanometers to about 2 nanometers.

5: The method of claims 2, 3 or 4, further comprising removing proteins from the processed sample.

6: The method of claim 5, wherein proteins are adsorbed to a substrate.

7: The method of claim 6, wherein the substrate is nitrocellulose, nylon or polyvinylidene fluoride.

8: The method of claims 2, 3, or 4 further comprising absorbing glucose from the processed sample.

9: The method of claim 8, wherein glucose is absorbed to a substrate consisting of porous absorbents having an internal surface area greater than about 400 M²/gram.

10: The method of claim 8, wherein glucose is absorbed to a substrate selected from the group consisting of: a zeolite, aluminum oxide microspheres, ceramic microspheres, hydrous alumina silicate microspheres, alumina dessicant beads, attapulgus clay beaded silica gel dessicants, natural clay absorbents, and activated carbon.

11: The method of claims 3, wherein the mammal is a human.

12: The method of claims 3, wherein the mammal is a companion animal.

13: The method of claim 12, wherein the companion animal is a cat or a dog.

14. The method of claim 1, wherein the mammal is afflicted with a disorder characterized by aberrant levels of blood carbohydrates.

15: The method of claim 14, wherein the disorder is diabetes.

16: The method of claim 15, wherein the quantities of salivary carbohydrates obtained from the processed sample indicate an appropriate therapeutic insulin dosage for treating the disorder.

17: The method of claim 1, wherein the mammal is preconditioned prior to obtaining the sample of saliva by being provided with a compound capable of stimulating the production and let down of saliva in the mammal.

18: A device for processing saliva comprising: a saliva sample introduction port, a filter and an absorbent matrix, wherein a sample of saliva is processed to remove high molecular weight contaminants and glucose in the processed saliva is absorbed to the matrix.

19: The device of claim 18, wherein the filter comprises tightly packed axially aligned fibers.

20: The device of claim 18, wherein the filter comprises one or more nanopore membranes, the nanopore membranes having a median pore diameter from about 200 nanometers to about 2 nanometers.

21: The device of claims 18, 19 or 20, further comprising a substrate capable of irreversibly binding proteins in the saliva sample.

22: The device of claim 21, wherein the substrate is nitrocellulose, nylon or polyvinylidene fluoride.

23: The device of claims 18, 19 or 20, further comprising a glucose absorbent substrate.

24: The device of claim 23, wherein the glucose absorbent substrate consists of porous absorbents having an internal surface area greater than about 400 M²/gram.

25: The device of claim 23, wherein the glucose absorbent substrate is selected from the group consisting of: a zeolite, aluminum oxide microspheres, ceramic microspheres, hydrous alumina silicate microspheres, alumina dessicant beads, attapulgus clay beaded silica gel dessicants, natural clay absorbents, and activated carbon.

26: The device of claim 18, further comprising a sensor for detecting glucose levels in the processed saliva sample.

27: The device of claim 8, further comprising a processor, wherein the processor correlates salivary carbohydrate levels in the sample with reference blood carbohydrate levels thereby calculating a range of probable blood carbohydrate levels based on the saliva sample carbohydrate levels and having an output for displaying information calculated by the processor.

28: The device of claim 27, wherein the processor correlates salivary carbohydrate levels of a user of the device with historical blood carbohydrate levels or historical salivary carbohydrate levels of the user of the device.

29: The device of claim 27, wherein the processor correlates salivary carbohydrate levels of a user of the device with historical medical or lifestyle information of the user of the device.

30: The device of claim 27, wherein the processor correlates salivary carbohydrate levels of a user of the device with genetic information about the user of the device.

31: The device of claim 30, wherein the output displays information indicating an appropriate therapeutic insulin dosage for the user.

32: The method of claim 9, wherein glucose is absorbed to a substrate selected from the group consisting of a zeolite, aluminum oxide microspheres, ceramic microspheres, hydrous alumina silicate microspheres, alumina dessicant beads, attapulgus clay beaded silica gel dessicants, natural clay absorbents, and activated carbon.

33: The method of claim 5, wherein the mammal is a human.

34: The method of claims 5, wherein the mammal is a companion animal.

35: The method of claim 34, wherein the companion animal is a cat or a dog.

36: The method of claim 8, wherein the mammal is a human.

37: The method of claims 8, wherein the mammal is a companion animal.

38: The method of claim 37, wherein the companion animal is a cat or a dog.

39: The device of claim 24, wherein the glucose absorbent substrate is selected from the group consisting of: a zeolite, aluminum oxide microspheres, ceramic microspheres, hydrous alumina silicate microspheres, alumina dessicant beads, attapulgus clay beaded silica gel dessicants, natural clay absorbents, and activated carbon.
